(19) United States
(12) Patent Application Publication
(10) Pub. No.: US 2025/0311993 A1
(43) Pub. Date: Oct. 9, 2025

(54) MEDICAL INFORMATION PROCESSING APPARATUS, MEDICAL INFORMATION PROCESSING METHOD, AND STORAGE MEDIUM

(71) Applicant: CANON MEDICAL SYSTEMS CORPORATION, Otawara-shi (JP)

(72) Inventor: Masakazu MATSUURA, Nasushiobara (JP)

(73) Assignee: CANON MEDICAL SYSTEMS CORPORATION, Otawara-shi (JP)

(21) Appl. No.: 19/097,140

(22) Filed: Apr. 1, 2025

(30) Foreign Application Priority Data
Apr. 3, 2024 (JP) 2024-059882

(51) Int. Cl. A61B 6/00; A61B 6/42; A61B 6/46

(52) U.S. Cl. CPC A61B 6/5258; A61B 6/4241; A61B 6/465; A61B 6/466

(57) ABSTRACT

A medical image diagnostic apparatus of an embodiment includes processing circuitry. The processing circuitry acquires projection data of X-rays with which a subject is irradiated. The processing circuitry generates a medical image from the projection data through reconstruction processing. The processing circuitry removes noise from the medical image using a trained model. The processing circuitry outputs a denoise image via an output interface. The trained model is a machine learning model that is trained on the basis of a training data set in which an output image including noise is associated as a target with an input image including noise.

### CROSS-REFERENCE TO RELATED APPLICATION

Priority is claimed on Japanese Patent Application No. 2024-059882, filed Apr. 3, 2024, the content of which is incorporated herein by reference.

### FIELD

An embodiment disclosed in the present specification and the drawings relates to a medical information processing apparatus, a medical information processing method, and a storage medium.

### BACKGROUND

In an X-ray computed tomography (CT) apparatus, in recent years, a noise reduction technique using deep learning has been actively proposed, and as a result, this has significantly contributed to radiation exposure reduction. Also, in dual energy (DE) or photon counting CT (PCCT) spectral imaging, it is necessary to always take into account radiation exposure reduction, and noise reduction is a necessary technique.

In many noise reduction techniques using deep learning, target data with less noise is required as a training data set for training a machine learning model such as a deep neural network. However, it is difficult to obtain data of high-dose imaging with less noise as target data due to an increase in radiation exposure. For this reason, the machine learning model may not be trained using an appropriate training data set, and as a result, noise may not be appropriately removed from a medical image and the image quality of the medical image may not be improved.

### BRIEF DESCRIPTION OF THE DRAWINGS

FIG. 1 is a diagram showing a configuration example of a medical information processing system in a first embodiment.

FIG. 2 is a diagram showing a configuration example of an X-ray CT apparatus in the first embodiment.

FIG. 3 is a flowchart illustrating an example of a flow of a series of processing of the X-ray CT apparatus in the first embodiment.

FIG. 4 is a diagram schematically showing a flow of a series of processing of the X-ray CT apparatus in the first embodiment.

FIG. 5 is a diagram showing a configuration example of a training apparatus in the first embodiment.

FIG. 6 is a flowchart illustrating a flow of a series of processing of the training apparatus according to the first embodiment.

FIG. 7 is a diagram illustrating details of generation of a noise image.

FIG. 8 is a diagram illustrating details of training of a machine learning model.

FIG. 9 is a diagram showing an example of a configuration of a DAS of an X-ray CT apparatus according to a second embodiment.

FIG. 10 is a diagram showing an example of a functional configuration of a reconstruction function according to the second embodiment.

### DETAILED DESCRIPTION

Hereinafter, a medical information processing apparatus, a medical information processing method, and a storage medium according to an embodiment will be described with reference to the drawings.

A medical image diagnostic apparatus of the embodiment includes processing circuitry. The processing circuitry acquires projection data of X-rays with which a subject is irradiated. The processing circuitry generates a medical image from the projection data through reconstruction processing. The processing circuitry removes noise from the medical image using a trained model. The processing circuitry outputs a denoise image that is the medical image with the noise removed, via an output interface. The trained model is a machine learning model that is trained on the basis of a training data set in which an output image including noise is associated as a target with an input image including noise. In this case, it is possible to improve the image quality of a medical image by performing denoising using a machine learning model trained on the basis of an appropriate training data set.

### First Embodiment

### Configuration of Medical System

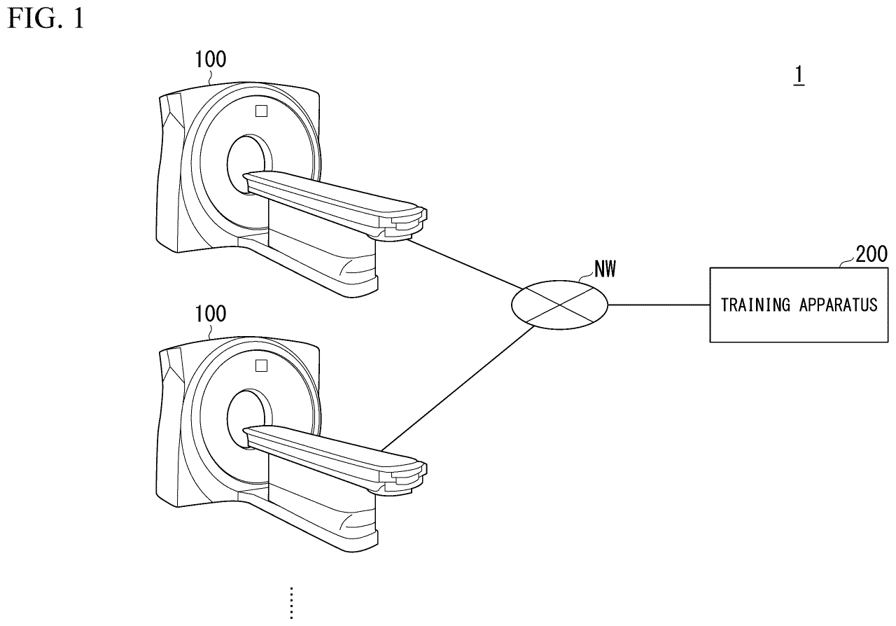

FIG. 1 is a diagram showing a configuration example of a medical information processing system 1 in a first embodiment. The medical information processing system 1 includes, for example, a plurality of medical image diagnostic apparatuses 100 and a training apparatus 200. The medical image diagnostic apparatuses 100 and the training apparatus 200 are connected in a communicative manner via a communication network NW.

The communication network NW may mean general information communication networks using telecommunication technology. For example, the communication network NW includes a telephone communication line network, an optical fiber communication network, a cable communication network, a satellite communication network, or the like, in addition to a wireless/wired local area network (LAN) such as a hospital backbone LAN or the Internet network.

The medical image diagnostic apparatus 100 is, for example, an X-ray computed tomography (CT) apparatus, and is typically provided in a medical institute, a research facility, or the like.

The training apparatus 200 receives information from each X-ray CT apparatus (medical image diagnostic apparatus) 100 via the communication network NW and trains a machine learning model MDL for removing noise from a medical image, using the received information. Then, the training apparatus 200 transmits the trained machine learning model MDL to each X-ray CT apparatus (medical image diagnostic apparatus) 100 via the communication network NW.

The training apparatus 200 may be a single apparatus or may be a system in which a plurality of apparatuses connected via the communication network NW operate in a cooperative manner. That is, the training apparatus 200 may be realized by a plurality of computers (processors) provided in a distributed computing system or a cloud computing system. The training apparatus 200 is not necessarily a separate apparatus different from the X-ray CT apparatus (medical image diagnostic apparatus) 100, and may be an apparatus integrated with the X-ray CT apparatus (medical image diagnostic apparatus) 100.

### Configuration of X-ray CT Apparatus

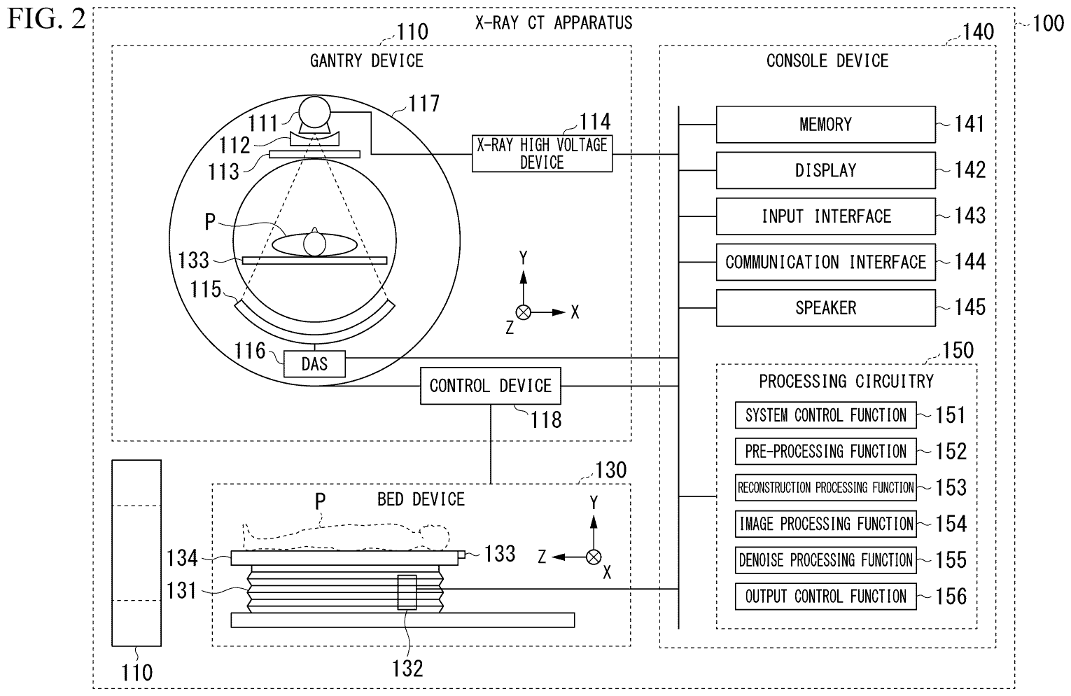

FIG. 2 is a diagram showing a configuration example of the X-ray CT apparatus 100 in the first embodiment. The X-ray CT apparatus 100 is an apparatus that generates a medical image (hereinafter, referred to as a CT image) of a subject P by scanning the subject P with X-rays, and diagnoses the subject P on the basis of the CT image. The subject P is typically a human; however, the subject P is not limited thereto, and may be other animals such as a dog or a cat or a plant. Hereinafter, an example where the subject P is a human will be described.

The X-ray CT apparatus 100 may be, for example, a CT apparatus using dual energy (DE) or photon counting CT (PCCT). In the first embodiment, a case where the X-ray CT apparatus 100 is a CT apparatus using dual energy (DE) will be described.

As shown in the drawing, the X-ray CT apparatus 100 includes, for example, a gantry device 110, a bed device 130, and a console device 140. In the example shown in the drawing, while a view of the gantry device 110 in a Z-axis direction and a view in an X-axis direction are shown for convenience of description, actually, there is only one gantry device 110. In the present embodiment, a rotation axis of a rotary frame 117 in a non-tilted state or a longitudinal direction of a top plate 133 of the bed device 130 is defined as the Z-axis direction, an axis that is perpendicular to the Z-axis direction and is horizontal to a floor surface is defined as the X-axis direction, and a direction that is perpendicular to the Z-axis direction and is a perpendicular to the floor surface is defined as a Y-axis direction.

The gantry device 110 has, for example, an X-ray tube 111, a wedge 112, a collimator 113, an X-ray high voltage device 114, an X-ray detector 115, a data acquisition system (hereinafter, referred to as DAS) 116, a rotary frame 117, and a control device 118.

The X-ray tube 111 generates X-rays by emitting thermoelectrons from a cathode (filament) toward an anode (target) with application of a high voltage from the X-ray high voltage device 114. The X-ray tube 111 includes a vacuum tube. For example, the X-ray tube 111 is a rotating anode type X-ray tube that generates X-rays by emitting thermoelectrons to a rotating anode.

The wedge 112 is a filter for adjusting the amount of X-rays with which the subject P is irradiated from the X-ray tube 111. The wedge 112 attenuates X-rays that pass therethrough such that the distribution of the amount of X-rays with which the subject P is irradiated from the X-ray tube 111 becomes a predetermined distribution. The wedge 112 is also called a wedge filter or a bow-tie filter. The wedge 112 is made of, for example, aluminum processed to have a prescribed target angle or a prescribed thickness.

The collimator 113 is a mechanism for narrowing down an irradiation range of the X-rays that pass through the wedge 112. The collimator 113 narrows down the irradiation range of the X-rays by forming a slit using a combination of a plurality of lead plates, for example. The collimator 113 may also be called an X-ray diaphragm.

The X-ray high voltage device 114 has, for example, a high-voltage generation device and an X-ray control device. The high-voltage generation device has an electric circuit including a transformer, a rectifier, and the like, and generates a high voltage that is applied to the X-ray tube 111. The X-ray control device controls an output voltage of the high-voltage generation device according to the amount of X-rays to be generated in the X-ray tube 111. The high-voltage generation device may boost a voltage using the above-described transformer or may boost a voltage using an inverter. The X-ray high voltage device 114 may be provided in the rotary frame 117 or may be provided in a fixed frame (not shown) of the gantry device 110.

The X-ray detector 115 detects the intensity of X-rays that are generated by the X-ray tube 111 and pass through and are incident on the subject P. The X-ray detector 115 outputs an electrical signal (an optical signal or the like) according to the detected intensity of the X-rays to the DAS 116. The X-ray detector 115 has, for example, a plurality of X-ray detection element rows. Each of the plurality of X-ray detection element rows has a plurality of X-ray detection elements arranged in a channel direction along an arc having a focal point of the X-ray tube 111 as a center. The plurality of X-ray detection element rows are arranged in a slice direction (row direction).

The X-ray detector 115 is, for example, an indirect detector having a grid, a scintillator array, and an optical sensor array. The scintillator array has a plurality of scintillators. Each scintillator has a scintillator crystal. The scintillator crystal emits light with an amount of light according to the intensity of incident X-rays. The grid is disposed on a surface of the scintillator array on which X-rays are incident, and has an X-ray shield plate having a function of absorbing scattered X-rays. The grid may also be called a collimator (one-dimensional collimator or two-dimensional collimator). The optical sensor array has, for example, optical sensors such as photomultiplier tubes (PMTs). The optical sensor array outputs an electrical signal according to an amount of light emitted by the scintillator. The X-ray detector 115 may be a direct conversion type detector having a semiconductor element that converts incident X-rays into an electrical signal.

The DAS 116 has, for example, an amplifier, an integrator, and an A/D converter. The amplifier performs amplification processing on an electrical signal output from each X-ray detection element of the X-ray detector 115. The integrator integrates the amplified electrical signal over a view period (described below). The A/D converter converts an electrical signal indicating an integration result into a digital signal. The DAS 116 outputs detection data based on the digital signal to the console device 140. The detection data is a digital value of the X-ray intensity identified by a channel number and a row number of the X-ray detection element as a generation source and a view number indicating a collected view. The view number is a number that changes according to the rotation of the rotary frame 117, and is, for example, a number that is incremented according to the rotation of the rotary frame 117. Accordingly, the view number is information indicating a rotation angle of the X-ray tube 111. The view period is a period that falls between a rotation angle corresponding to a certain view number and a rotation angle corresponding to a next view number. The DAS 116 may detect switching of views using a timing signal input from the control device 118, an internal timer, or a signal acquired from a sensor (not shown). In full scanning, during continuous exposure to X-rays from the X-ray tube 111, the DAS 116 collects a detection data group for the entire circumference (for 360 degrees). In half scanning, during continuous exposure to X-rays from the X-ray tube 111, the DAS 116 collects detection data for half the circumference (for 180 degrees).

The rotary frame 117 is an annular rotary member that rotates the X-ray tube 111, the wedge 112, and the collimator 113, and the X-ray detector 115 in a state in which these face each other. The rotary frame 117 is rotatably supported by a fixed frame around the subject P introduced therein. The rotary frame 117 further supports the DAS 116. The detection data output from the DAS 116 is transmitted from a transmitter having a light-emitting diode (LED) provided in the rotary frame 117 to a receiver having a photodiode provided in a non-rotating part (for example, a fixed frame) of the gantry device 110 through optical communication and is transferred to the console device 140 by the receiver. A transmission method of the detection data from the rotary frame 117 to the non-rotating part is not limited to the above-described method using optical communication, and any non-contact transmission method may be employed. The rotary frame 117 is not limited to an annular member and may be a member such as an arm as long as the member can support and rotate the X-ray tube 111 and the like.

The control device 118 has, for example, processing circuitry having a processor such as a central processing unit (CPU) and a drive mechanism including a motor, an actuator, and the like. The control device 118 receives an input signal from an input interface 143 attached to the console device 140 or the gantry device 110 and controls the operation of the gantry device 110 and the bed device 130.

The control device 118 rotates the rotary frame 117, tilts the gantry device 110, or moves the top plate 133 of the bed device 130, for example. In tilting the gantry device 110, the control device 118 rotates the rotary frame 117 around an axis parallel to the Z-axis direction on the basis of an inclination angle (tilt angle) input to the input interface 143. The control device 118 acquires a rotation angle of the rotary frame 117 on the basis of an output of a sensor (not shown), or the like. The control device 118 outputs the rotation angle of the rotary frame 117 to processing circuitry 150 at any time. The control device 118 may be provided in the gantry device 110 or may be provided in the console device 140.

The control device 118 causes the gantry device 110 to perform main scan imaging or perform scan imaging that is positioning imaging to be performed before execution of main scan imaging.

The bed device 130 is a device that introduces the subject P placed thereon as a scanning target into the rotary frame 117 of the gantry device 110. The bed device 130 has, for example, a base 131, a bed drive device 132, a top plate 133, and a support frame 134. The base 131 includes a housing that supports the support frame 134 to be movable in a vertical direction (Y-axis direction). The bed drive device 132 includes a motor and an actuator. The bed drive device 132 moves the top plate 133 on which the subject P is placed, in the longitudinal direction (Z-axis direction) of the top plate 133 along the support frame 134. The top plate 133 is a plate-shaped member on which the subject P is placed.

The console device 140 has, for example, a memory 141 (storage circuit), a display 142, an input interface 143, a communication interface 144, a speaker 145, and processing circuitry 150. In the present embodiment, while a case where the console device 140 is provided separately from the gantry device 110 will be described, some or all components of the console device 140 may be included in the gantry device 110.

The memory 141 is realized by, for example, a semiconductor memory element such as a random access memory (RAM) or a flash memory, a hard disk, or an optical disc. A storage medium such as a read only memory (ROM) or a register may be included in the memory 141.

The memory 141 stores, for example, detection data, projection data, or a reconstructed image. Such data may be stored in an external memory (for example, a network attached storage (NAS)) with which the X-ray CT apparatus 100 can communicate, instead of (or in addition to) the memory 141.

Model definition data is further stored in the memory 141. The model definition data is data such as a program or an algorithm that defines a trained model MDL described below.

The display 142 displays various kinds of information. For example, the display 142 displays a CT image generated by the processing circuitry 150 or a graphical user interface (GUI) that receives various operations from an operator. The operator is, for example, a medical worker, such as a doctor, an engineer, or a nurse. The display 142 is, for example, a liquid crystal display, a CRT, or an organic electro-luminescence (EL) display. The display 142 is an example of an “output interface”.

The input interface 143 receives various input operations from the operator and outputs electrical signals indicating the contents of the received input operations to the processing circuitry 150.

For example, the input interface 143 is realized by a pointing device (a mouse, a touch panel, a trackball, a joystick, a pen tablet, a stylus, or the like), a keyboard, a switch, a button, a foot pedal, a camera, an infrared sensor, a microphone, or the like. In the present specification, the input interface 143 is not limited to a configuration including a physical operation component such as a mouse or a keyboard. For example, electrical signal processing circuitry that receives an electrical signal corresponding to an input operation from an external input device provided separately from the device and outputs the electrical signal to a control circuit is also included as an example of the input interface 143.

The communication interface 144 includes, for example, a network interface card (NIC) or a wireless communication module. The communication interface 144 communicates with an external apparatus such as the training apparatus 200 via the communication network NW. The communication interface 144 is another example of an “output interface”.

The speaker 145 outputs sound on the basis of information output from the processing circuitry 150.

The processing circuitry 150 controls the operation of the entire X-ray CT apparatus 100. The processing circuitry 150 executes, for example, a system control function 151, a pre-processing function 152, a reconstruction function 153, an image processing function 154, a denoise processing function 155, and an output control function 156. The processing circuitry 150 realizes such functions by a hardware processor executing a program stored in the memory 141, for example.

The above-described DAS 116 and the pre-processing function 152 are an example of an “acquisition unit”. The reconstruction function 153 is an example of a “reconstruction unit”, the denoise processing function 155 is an example of a “denoise processing unit”, and the output control function 156 is an example of an “output control unit”.

The hardware processor means, for example, circuitry such as a central processing unit (CPU), a graphical processing unit (GPU), an application specific integrated circuit (ASIC), or a programmable logic device (for example, a simple programmable logic device (SPLD), a complex programmable logic device (CPLD), or a field programmable gate array (FPGA)).

The program may be directly incorporated into the circuit of the hardware processor, instead of being stored in the memory 141. In this case, the hardware processor realizes the functions by reading out and executing the program incorporated into the circuit.

The above-described program may be stored in the memory 141 in advance or may be stored in a non-transitory storage medium such as a DVD or a CD-ROM and may be installed to the memory 141 from the non-transitory storage medium when the non-transitory storage medium is loaded into a drive device (not shown) of the console device 140.

The hardware processor is not limited to a configuration as a single circuit and may be configured as a single hardware processor by combining a plurality of independent circuits to realize each function. A plurality of components may be integrated into a single hardware processor to realize each function.

The components in the console device 140 or the processing circuitry 150 may be distributed and realized by a plurality of hardware components. The processing circuitry 150 may be realized by an external apparatus (for example, the training apparatus 200) that can communicate with the console device 140, instead of the configuration in the console device 140. The external device may be, for example, a workstation connected to a single X-ray CT apparatus 100 or may be an apparatus (for example, a cloud server) that is connected to a plurality of X-ray CT apparatuses 100 and collectively executes the same processing as those in the processing circuitry 150.

The system control function 151 controls various functions of the processing circuitry 150 on the basis of an operation input to the input interface 143. The system control function 151 acquires information from the external apparatus such as the training apparatus 200 via the communication interface 144.

The pre-processing function 152 performs pre-processing on detection data output from the DAS 116 to generate projection data and stores the generated projection data in the memory 141. The pre-processing includes, for example, various kinds of processing such as logarithmic conversion processing, offset correction processing, inter-channel sensitivity correction processing, and beam hardening correction.

The reconstruction function 153 performs reconstruction processing using a filtered back projection method, a successive approximation reconstruction method, or the like on the projection data generated by the pre-processing function 152 to generate a CT image (also referred to as a reconstructed image) and stores the generated CT image (reconstructed image) in the memory 141.

The image processing function 154 converts the CT image (including a denoised CT image described below) into a three-dimensional image or cross-section data of any cross section using a known method on the basis of an operation input to the input interface 143. The conversion into the three-dimensional image may be performed by the pre-processing function 152.

The denoise processing function 155 removes noise from the CT image (reconstructed image) generated by the reconstruction function 153 through the reconstruction processing. For example, the denoise processing function 155 may remove noise from the CT image using a machine learning model MDL trained in advance (hereinafter, also referred to as a trained model MDL). MDL is a simple symbol that means a model. Details of denoise processing using the trained model MDL will be described below.

The output control function 156 outputs various kinds of information by controlling the display 142, the input interface 143, the communication interface 144, and the speaker 145.

For example, the output control function 156 may display the CT image subjected to denoising by the denoise processing function 155 on the display 142 or may display the CT image before denoising by the denoise processing function 155 on the display 142. The output control function 156 may display, on the display 142, a graphical user interface (GUI) that receives various operations from the operator such as a doctor or an engineer.

### Processing Flow/Runtime (Inference) of X-ray CT Apparatus

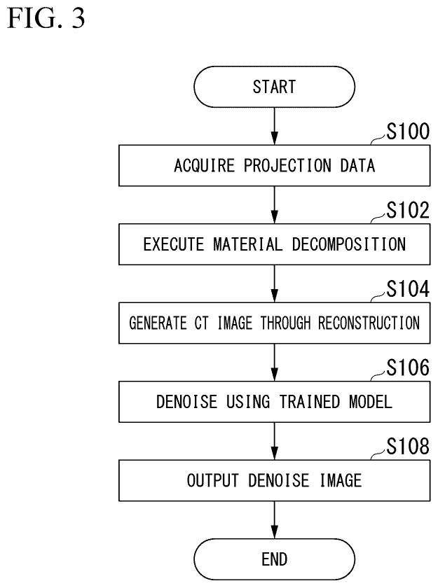

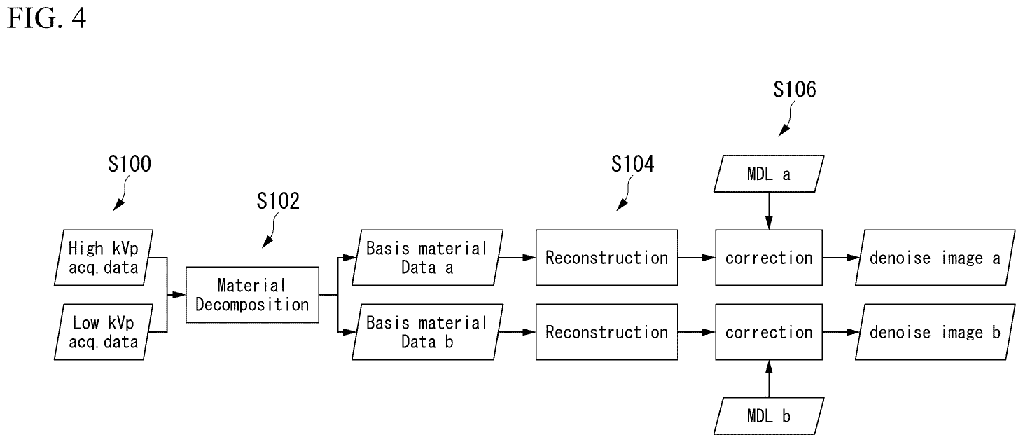

Hereinafter, a flow of a series of processing of the X-ray CT apparatus 100 will be described with reference to FIGS. 3 and 4. FIG. 3 is a flowchart illustrating an example of a flow of a series of processing of the X-ray CT apparatus 100 in the first embodiment. FIG. 4 is a diagram schematically showing a flow of a series of processing of the X-ray CT apparatus 100 in the first embodiment. The processing of the flowchart is executed after the machine learning model MDL is trained.

First, the pre-processing function 152 performs pre-processing on detection data output from the DAS 116 to acquire projection data (Step S100).

The X-ray CT apparatus 100 according to the first embodiment is a DECT apparatus and images the subject P with two types of X-rays having different tube voltages. For this reason, high tube voltage (kVp is equal to or greater than a threshold value) projection data and low tube voltage (kVp is less than the threshold value) projection data are included in the projection data. The threshold value is, for example, about 120 kVp.

Next, the reconstruction function 153 performs material decomposition on the basis of the acquired high-tube voltage (high kVp) projection data and low-tube voltage (low kVp) projection data (Step S102).

For example, the reconstruction function 153 may perform material decomposition on two types of projection data using attenuation coefficients of two different types of basis materials (also referred to as basis materials) a and b such as iodine and water. The number of basis materials is not limited to two and may be one or may be three or more.

Next, the reconstruction function 153 performs reconstruction processing on the material-decomposed projection data to generate a CT image (also referred to as a basis material image) in which each basis material is enhanced or suppressed (Step S104).

For example, the reconstruction function 153 performs reconstruction processing on projection data in which the basis material a is decomposed, to generate a CT image in which the basis material a (for example, iodine) is enhanced. Similarly, the reconstruction function 153 performs reconstruction processing on projection data in which the basis material b is decomposed, to generate a CT image in which the basis material b (for example, water) is enhanced. Hereinafter, description will be provided while the CT image in which the basis material a is enhanced is referred to as a “basis material image a”, and the CT image in which the basis material b is enhanced is referred to as a “basis material image b”. The projection data in which basis material a is decomposed and the projection data in which the basis material b is decomposed are an example of “basis material projection data”.

Next, the denoise processing function 155 removes noise from the CT images (here, basis material images) generated through the reconstruction processing of the reconstruction function 153, using the trained model MDL (Step S106).

For example, the denoise processing function 155 reads out the trained model MDL defined by the model definition data stored in the memory 141 and inputs the CT image generated through the reconstruction processing to the trained model MDL.

The trained model MDL may be implemented by, for example, a deep neural network such as a convolutional neural network(s) (CNN). The trained model MDL may be implemented using other machine learning models such as a support vector machine, a decision tree, a random forest, and logistics regression, instead of the neural network.

When the trained model MDL is implemented by the neural network, for example, coupling information representing a method of coupling units included in an input layer, one or more hidden layers (intermediate layers), and an output layer that configure the neural network, weight information representing a coupling coefficient given to data input and output between coupled units, and the like are included in the model definition data.

The coupling information includes, for example, the number of units included in each layer, information for designating a type of a unit that is a coupling destination of each unit, and information such as an activation function that realizes each unit and a gate provided between the units of the hidden layers.

The activation function that realizes the unit may be, for example, a rectified linear unit (ReLU) function, an exponential linear units (ELU) function, a clipping function, a Sigmoid function, a step function, a hyperbolic tangent function, or an identify function. The gate selectively transmits or weights data transferred between the units according to a value (for example, 1 or 0) returned by the activation function.

The coupling coefficient includes, for example, a weight given to output data when data is output from a unit of a certain layer to a unit of a deeper layer in a hidden layer of the neural network. The coupling coefficient may include a bias component or the like unique to each layer.

When the projection data is decomposed into two types of basis materials a and b in the process of the reconstruction processing, two types of models are included in the trained model MDL along with the basis materials a and b. That is, a first trained model MDLa trained in advance assuming that the basis material image a is input and a second trained model MDLb trained in advance assuming that the basis material image b is input are included.

The two types of trained models MDL are trained to output, when each basis material image is input, a CT image (that is, a denoise image) with noise reduced from the image. Accordingly, the denoise processing function 155 acquires the denoise image of the basis material image a from the first trained model MDLa by inputting the basis material image a to the first trained model MDLa. Similarly, the denoise processing function 155 acquires the denoise image of the basis material image b from the second trained model MDLb by inputting a CT image with the basis material image b enhanced to the second trained model MDLb. The basis material image a is an example of a “first medical image”, and the basis material image b is an example of a “second medical image”.

Next, the output control function 156 outputs the denoise images (Step S108). For example, the output control function 156 may display the denoise image of the basis material a and the denoise image of the basis material b on the display 142. The output control function 156 may transmit the denoise images to an external apparatus (for example, a computer that is used by a doctor, an engineer, or the like) via the communication interface 144. Accordingly, the processing of this flowchart ends.

### Configuration of Training Apparatus

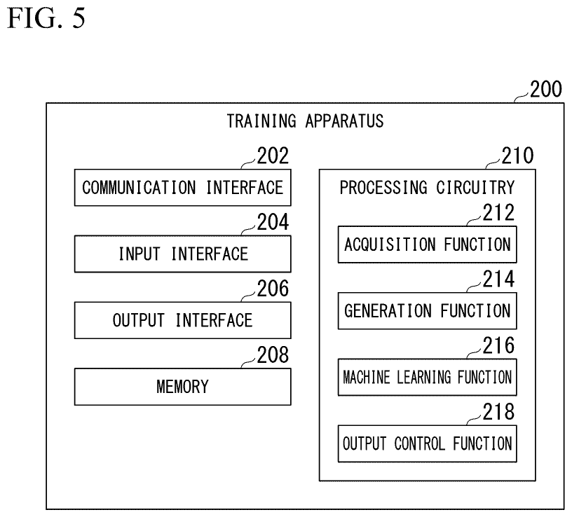

Hereinafter, the configuration of the training apparatus 200 that trains the machine learning model MDL will be described. FIG. 5 is a diagram showing a configuration example of the training apparatus 200 in the first embodiment. The training apparatus 200 includes, for example, a communication interface 202, an input interface 204, an output interface 206, a memory 208, and processing circuitry 210.

The communication interface 202 communicates with an external apparatus via the communication network NW. The communication interface 202 includes, for example, an NIC and an antenna for wireless communication.

The input interface 204 receives various input operations from the operator, converts the received input operations into electrical signals, and outputs the electrical signals to the processing circuitry 210.

For example, the input interface 204 includes a mouse, a keyboard, a trackball, a switch, a button, a joystick, or a touch panel. The input interface 204 may be, for example, a user interface such as a microphone that receives a voice input. When the input interface 204 is a touch panel, the input interface 204 may also have a display function of a display 213a included in the output interface 206 described below.

In the present specification, the input interface 204 is not limited to a configuration including a physical operation component such as a mouse or a keyboard. For example, electrical signal processing circuitry that receives an electrical signal corresponding to an input operation from an external input device provided separately from the apparatus and outputs the electrical signal to a control circuit is also included as an example of the input interface 204.

The output interface 206 includes, for example, a display 213a and a speaker 213b. The display 213a displays various kinds of information.

For example, the display 213a displays an image generated by the processing circuitry 210 or a GUI for receiving various input operations from the operator. For example, the display 213a is an LCD, a CRT display, or an organic EL display. The speaker 213b outputs information input from the processing circuitry 210 as sound.

The memory 208 is realized by, for example, a semiconductor memory element such as a RAM or a flash memory, a hard disk, or an optical disc. The non-transitory storage medium may be realized by other storage devices such as an NAS or an external storage server apparatus connected via the communication network NW.

The memory 208 may include a non-transitory storage medium such as a ROM or a register. In the memory 208, a program that is executed by a hardware processor of the processing circuitry 210, various calculation results of the processing circuitry 210, model definition data, and the like are stored.

The processing circuitry 210 includes, for example, an acquisition function 212, a generation function 214, a machine learning function 216, and an output control function 218.

The processing circuitry 210 realizes such functions by the hardware processor (computer) executing the program stored in the memory 208 (storage circuit), for example.

The hardware processor in the processing circuitry 210 means, for example, circuitry such as a CPU, a GPU, an application specific integrated circuit, or a programmable logic device (for example, a simple programmable logic device, a complex programmable logic device, or a field programmable gate array).

The program may be incorporated into the circuit of the hardware processor, instead of being stored in the memory 208. In this case, the hardware processor realizes the functions by reading out and executing the program incorporated into the circuit. The above-described program may be stored in the memory 208 in advance or may be stored in a non-transitory storage medium, such as a DVD or a CD-ROM and may be installed to the memory 208 from the non-transitory storage medium when the non-transitory storage medium is loaded into a drive device (not shown) of the training apparatus 200.

The hardware processor is not limited to a configuration as a single circuit and may be configured as a single hardware processor by combining a plurality of independent circuits to realize each function. A plurality of components may be integrated into a single hardware processor to realize each function.

### Processing Flow/Training of Training Apparatus

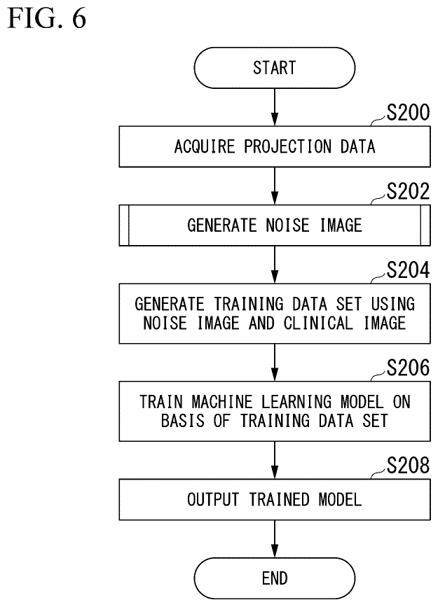

Hereinafter, processing of training of the training apparatus 200 will be described with reference to a flowchart. FIG. 6 is a flowchart illustrating a flow of a series of processing of the training apparatus 200 according to the first embodiment. The processing of this flowchart is executed in training the machine learning model MDL.

First, the acquisition function 212 acquires high-tube voltage (high kVp) projection data and low-tube voltage (low kVp) projection data (Step S200).

For example, the acquisition function 212 may access the X-ray CT apparatus 100 via the communication interface 202 and may acquire the projection data from the X-ray CT apparatus 100. When the user inputs the projection data to the input interface 204, the acquisition function 212 may acquire the projection data from the input interface 204. When the projection data is stored in the memory 208, the acquisition function 212 may acquire the projection data from the memory 208.

Next, the generation function 214 generates noise images from the projection data acquired by the acquisition function 212 (Step S202).

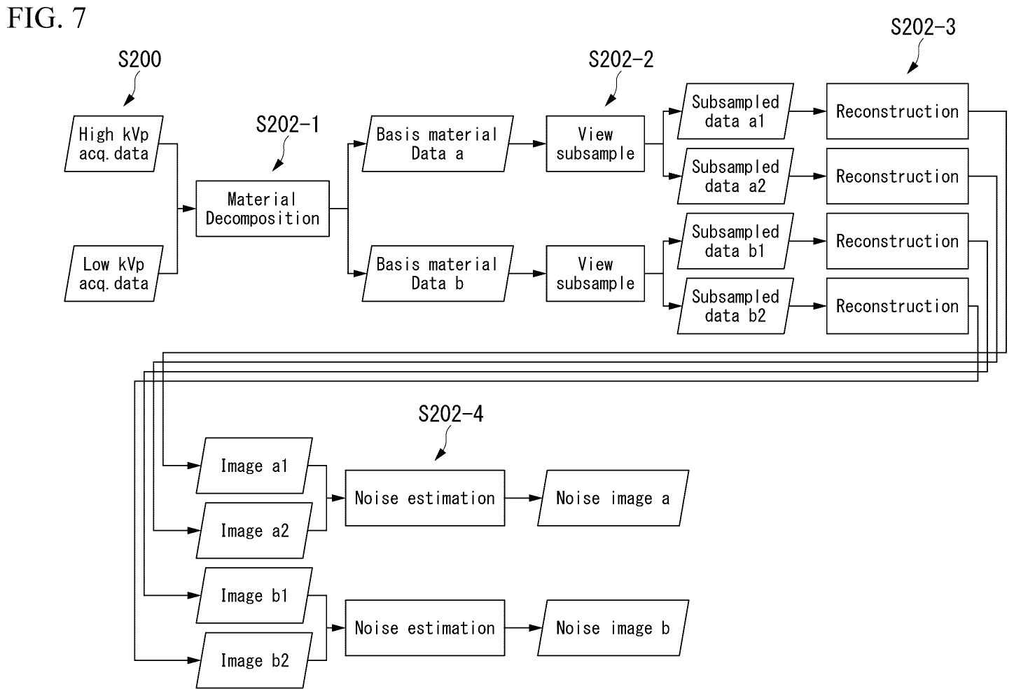

FIG. 7 is a diagram illustrating details of generation of a noise image. When the high-tube voltage (high kVp) projection data and the low-tube voltage (low kVp) projection data are acquired in the processing of S200, similarly to the processing of S102 described above, the generation function 214 performs material decomposition on the basis of the high-tube voltage (high kVp) projection data and the low-tube voltage (low kVp) projection data (Step S202-1).

For example, as described above, when the material decomposition is performed using the attenuation coefficients of two different types of basis materials a and b, the generation function 214 generates projection data in which the basis material a is decomposed and projection data in which the basis material b is decomposed.

Next, the generation function 214 separates each piece of projection data into two pieces of data by subsampling each of the two types of projection data (Step S202-2).

For example, the generation function 214 may generate subsampled data a1 and a2 from the projection data of the basis material a and may generate subsampled data b1 and b2 from the projection data of the basis material b, by subsampling. Te projection data of the basis material a and the projection data of the basis material b are an example of “training projection data”.

For example, when the projection data of the basis material a has the number of pieces of data of 1200 views, the generation function 214 may assign data for even-numbered 600 views to the subsampled data a1 and may assign data for odd-numbered 600 views to the subsampled data a2. The same applies to the projection data of the basis material b. The subsampled data a1 is an example of “first subsampled data”, and the subsampled data a2 is an example of “second subsampled data”.

The generation function 214 may sample the projection data with the channels or the rows of the X-ray detector 115 or may sample the projection data randomly, instead of sampling the projection data with the number of views. The two pieces of subsampled data separated from one piece of projection data are not necessarily completely independent and may partially overlap each other.

Next, the generation function 214 performs the reconstruction processing on each piece of subsampled data to generate a CT image (basis material image) in which each basis material is enhanced or suppressed (Step S202-3). In the reconstruction, reconstruction to an image type in spectral imaging may be included in addition to back projection reconstruction.

For example, the generation function 214 generates the basis material image a1 derived from the subsampled data a1 by reconstructing the subsampled data a1, and generates the basis material image a2 derived from the subsampled data a2 by reconstructing the subsampled data a2. Similarly, the generation function 214 generates the basis material image b1 derived from the subsampled data b1 by reconstructing the subsampled data b1, and generates the basis material image b2 derived from the subsampled data b2 by reconstructing the subsampled data b2. The basis material image a1 derived from the subsampled data a1 and the basis material image b1 derived from the subsampled data b1 are an example of a “first training image”, and the basis material image a2 derived from the subsampled data a2 and the basis material image b2 derived from the subsampled data b2 are an example of a “second training image”.

Next, the generation function 214 generates noise images from a plurality of basis material images (Step S202-4).

For example, the generation function 214 generates a noise image of the basis material a according to Expression (1), and generates a noise image of the basis material b according to Expression (2).

$$\begin{matrix}[ Equation 1 ] & \\ Noise image a = α 1 ( a 1 - a 2 ) & ( 1 )\end{matrix}$$

$$\begin{matrix}[ Equation 2 ] & \\ Noise image b = β 1 ( b 1 - b 2 ) & ( 2 )\end{matrix}$$

α1 and β1 are a weight coefficient that adjusts a noise level. The weight coefficients α1 and β1 may be values different from each other.

The noise image of the basis material a is calculated by multiplying a difference (a1-a2) between the basis material image a1 derived from the subsampled data a1 and the basis material image a2 derived from the subsampled data a2 by the weight coefficient a1 as Expression (1). The noise image of the basis material b is calculated by multiplying a difference (b1-b2) between the basis material image b1 derived from the subsampled data b1 and the basis material image b2 derived from the subsampled data b2 by the weight coefficient β1 as Expression (2).

Here, returning to description of the flowchart of FIG. 6. Next, when the noise images are generated, the machine learning function 216 generates a training data set using the noise images and clinical images described below (Step S204).

Next, the machine learning function 216 trains an machine learning model MDL in an untrained state using the training data set (Step S206). The untrained state may be a state of having never been trained or may be a state of being trained several times but insufficiently trained.

Next, the output control function 218 transmits the model definition data that defines the machine learning model MDL trained by the machine learning function 216, that is, the trained model MDL, to each X-ray CT apparatus 100 via the communication interface 202 (Step S208). Accordingly, the processing of this flowchart ends.

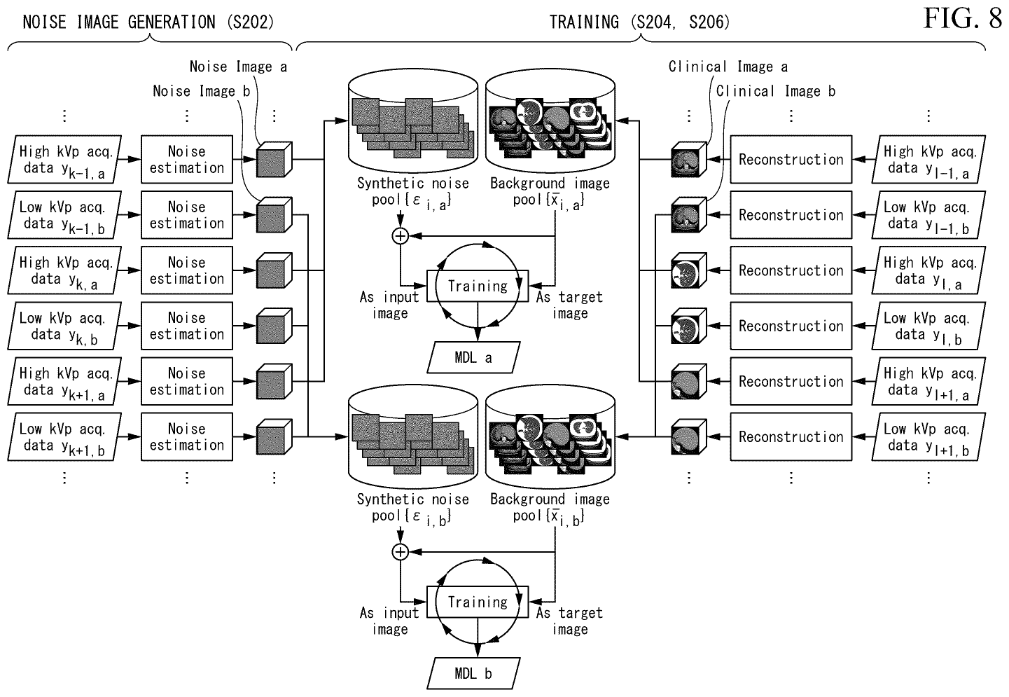

FIG. 8 is a diagram illustrating details of training of the machine learning model MDL. Processing on the left side in the drawing corresponds to the processing (that is, noise image generation) of S202 described above, and processing on the right side in the drawing corresponds to the processing of S204 and S206.

The machine learning function 216 accumulates the noise image of the basis material a among a plurality of noise images generated in the processing of S202 in a first synthetic noise pool {ε_{i,a}}, and accumulates the noise image of the basis material b in a second synthetic noise pool {ε_{i,b}}. A storage region of the synthetic noise pools is secured in the memory 208.

On the other hand, the machine learning function 216 generates a clinical basis material image a and a clinical basis material image b by reconstructing the projection data of the basis material a and the projection data of the basis material b acquired at a timing (for example, regularly in the background) different from S200. Various kinds of noise caused by scanning are included in the clinical basis material images a and b.

The machine learning function 216 accumulates the basis material image a among a plurality of basis material images in a first background image pool {x\hat{ }-_{i,a}}, and accumulates the basis material image b in a second background image pool {x\hat{ }-_{i,b}}. A storage region of the background image pools is also secured in the memory 208.

The machine learning function 216 generates an image (hereinafter, referred to as a synthetic image a) in which the noise image of the basis material a accumulated in the first synthetic noise pool {ε_{i,a}} and the basis material image a accumulated in the first background image pool {x\hat{ }-_{i,a}} are added. Next, the machine learning function 216 generates a first training data set in which the basis material image a added to the noise image of the basis material a is associated as a target (also referred to as a label) with the synthesized image a.

Then, the machine learning function 216 trains a first machine learning model MDLa in an untrained state on the basis of the first training data set. Specifically, the machine learning function 216 inputs the synthetic image a included as input data in the first training data set to the first machine learning model MDLa in the untrained state. The machine learning function 216 calculates a difference between an image output from the first machine learning model MDLa with the input of the synthetic image a and the basis material image a included as target data (output data) in the first training data set, and adjusts parameters (weight coefficient, bias component, and the like) of the first machine learning model MDLa such that the difference decreases.

On the other hand, the machine learning function 216 generates an image (hereinafter, referred to as a synthetic image b) in which the noise image of the basis material b accumulated in the second synthetic noise pool {ε_{i,b}} and the basis material image b accumulated in the second background image pool {x\hat{ }-_{1,b}} are added. Next, the machine learning function 216 generates a second training data set in which the basis material image b added to the noise image of the basis material b is associated as a target with the synthetic image b.

Then, the machine learning function 216 trains a second machine learning model MDLb in an untrained state on the basis of the second training data set. Specifically, the machine learning function 216 inputs the synthetic image b included as input data in the second training data set to the second machine learning model MDLb in the untrained state. The machine learning function 216 calculates a difference between an image output from the second machine learning model MDLb with the input of the synthetic image b and the basis material image b included as target data (output data) in the second training data set, and adjusts parameters (weight coefficient, bias component, and the like) of the second machine learning model MDLb such that the difference decreases.

The machine learning function 216 repeats the above-described processing until the number of iterations of training reaches a specified number, and when the number of iterations reaches the specified number, stores model definition data that defines the first trained model MDLa and the second trained model MDLb, in the memory 208. With the execution of the series of training, the model definition data in which the first trained model MDLa and the second trained model MDLb are defined is provided to each X-ray CT apparatus 100.

According to the first embodiment described above, the X-ray CT apparatus 100 acquires the projection data of the X-rays with which the subject P is irradiated, and generates the CT image from the projection data through the reconstruction processing. Then, the X-ray CT apparatus 100 removes noise from the CT image using the trained model MDL, and displays the denoise image that is the CT image with noise removed, on the display 142 or transmits the denoise image to an external apparatus via the communication interface 144.

The trained model MDL is trained using the training data set in which an output image including noise similarly is associated as a target with an input image including noise.

In general, for denoising, an image (that is, clean image) including no noise or less noise is employed as a target image included in the training data set. This is a method called Noise2Clean.

In contrast, there is a method called Noise2Noise. This is a method employing an image including noise as a target image included in the training data set, and it is said that noise can be removed with accuracy comparable to Noise2Clean.

The method of the present embodiment is conceived from Noise2Noise, and generates, for example, a training data set in which a basis material image (an image including noise that naturally occurs in clinical practice) is associated as a target with a synthetic image (that is, an image in which both noise obtained through calculation and noise that naturally occurs in clinical practice are added) in which a noise image of a basis material and the basis material image are added. In this way, it is possible to generate a machine learning model MDL that, even when an image with noise is employed as a target image, can remove noise with high accuracy similarly to when high-dose projection data with less noise is employed. Furthermore, since noise is removed from the medical image using the machine learning model MDL trained on the basis of Noise2Noise, it is possible to improve the image quality of the medical image.

### Modification Example of First Embodiment

Hereinafter, a modification example of the first embodiment will be described. In the first embodiment described above, while a case where the medical image generated through the reconstruction processing is the basis material image has been described, the present disclosure is not limited thereto. For example, a virtual monochromatic X-ray image, a virtual simple image, an iodine map, an effective atomic number image, or an electron density image may be generated through the reconstruction processing. In this case, it is assumed that a medical image included in each training data set is also a virtual monochromatic X-ray image, a virtual simple image, an iodine map, an effective atomic number image, or an electron density image.

In the first embodiment described above, as shown in FIG. 7, while a case where the noise image is generated from the difference between two different basis material images has been described, the present disclosure is not limited thereto. For example, the generation function 214 may collect projection data of an object with a homogeneous material such as a water phantom or air and may generate a noise image on the basis of the projection data. The generation function 214 may calculate noise through a simulation.

### Second Embodiment

Hereinafter, a second embodiment will be described. In the first embodiment described above, a case where the X-ray CT apparatus 100 is a CT apparatus using dual energy (DE) has been described. In contrast, the second embodiment is different from the first embodiment in that the X-ray CT apparatus 100 is a photon counting CT (PCCT) apparatus. Hereinafter, description will be provided focusing differences from the first embodiment, and description of common points to the first embodiment will not be repeated. In the description of the second embodiment, the same parts as those in the first embodiment are represented by the same reference numerals.

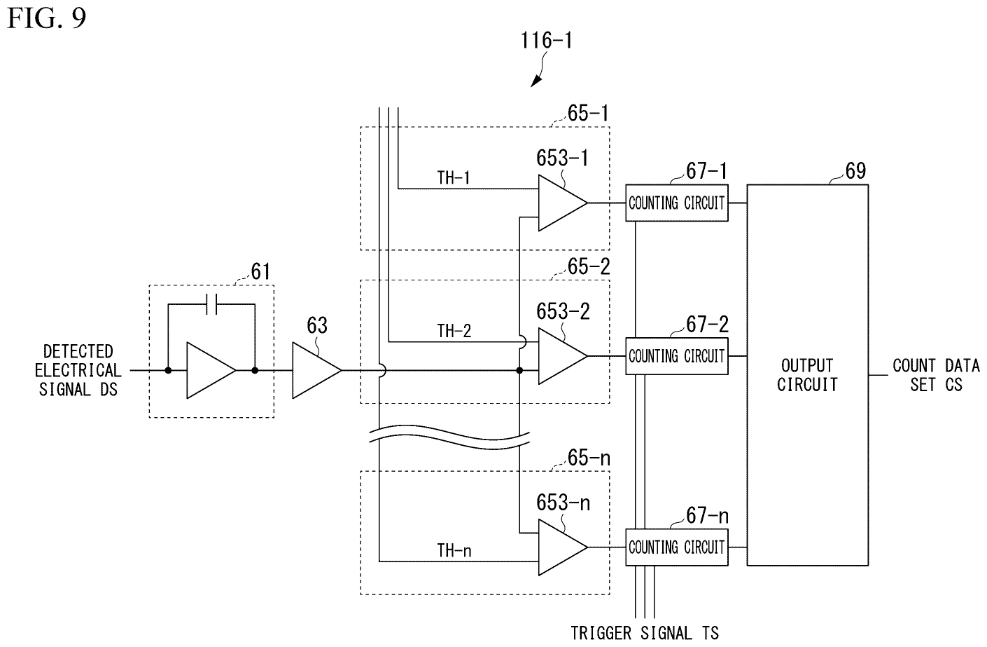

FIG. 9 is a diagram showing an example of a configuration of a DAS 116 of an X-ray CT apparatus 100 according to the second embodiment. The DAS 116 of the X-ray CT apparatus 100, which is a photon counting CT apparatus, includes readout channels for the number of channels corresponding to the number of X-ray detection elements. A plurality of readout channels are implemented in parallel on an integrated circuit such as an application specific integrated circuit (ASIC). FIG. 9 shows only a configuration of a DAS 116-1 for one readout channel.

The DAS 116-1 has a preamplifier circuit 61, a waveform shaping circuit 63, a plurality of pulse height discrimination circuits 65, a plurality of counting circuits 67, and an output circuit 69. The preamplifier circuit 61 amplifies a detected electrical signal DS (current signal) from an X-ray detection element that is a connection destination. For example, the preamplifier circuit 61 converts the current signal from the X-ray detection element that is a connection destination, into a voltage signal having a voltage value (pulse height value) proportional to the amount of change of the current signal. The waveform shaping circuit 63 is connected to the preamplifier circuit 61. The waveform shaping circuit 63 shapes the waveform of the voltage signal of the preamplifier circuit 61. For example, the waveform shaping circuit 63 reduces a pulse width of the voltage signal of the preamplifier circuit 61.

A plurality of counting channels corresponding to the number of energy bands (energy bins) are connected to the waveform shaping circuit 63. When n energy bins are set, the waveform shaping circuit 63 is provided with n counting channels. Each counting channel has a pulse height discrimination circuit 65-n and a counting circuit 67-n.

Each of the pulse height discrimination circuits 65-n discriminates the energy of X-ray photons detected by the X-ray detection element, which is the pulse height value of the voltage signal from the waveform shaping circuit 63. For example, the pulse height discrimination circuit 65-n has a comparison circuit 653-n. The voltage signal from the waveform shaping circuit 63 is input to one input terminal of each of the comparison circuits 653-n. Reference signals TH (reference voltage value) corresponding to different threshold values are supplied from the control device 118 to the other input terminals of the respective comparison circuits 653-n. For example, a reference signal TH-1 is supplied to the comparison circuit 653-1 for an energy bin bin1, a reference signal TH-2 is supplied to the comparison circuit 653-2 for an energy bin bin2, and a reference signal TH-n is supplied to the comparison circuit 653-n for an energy bin binn. Each of the reference signals TH has an upper limit reference value and a lower limit reference value. When the voltage signal from the waveform shaping circuit 63 has a pulse height value that corresponds to the energy bin corresponding to each reference signal TH, each of the comparison circuits 653-n outputs an electrical pulse signal. For example, when the pulse height value of the voltage signal from the waveform shaping circuit 63 is a pulse height value corresponding to the energy bin bin1 (when the pulse height value is a value between the reference signals TH-1 and TH-2), the comparison circuit 653-1 outputs an electrical pulse signal. On the other hand, when the pulse height value of the voltage signal from the waveform shaping circuit 63 is not the pulse height value corresponding to the energy bin bin1, the comparison circuit 653-1 for the energy bin bin1 does not output an electrical pulse signal. For example, when the pulse height value of the voltage signal from the waveform shaping circuit 63 is a pulse height value corresponding to the energy bin bin2 (when the pulse height value is a value between the reference signals TH-2 and TH-3), the comparison circuit 653-2 outputs an electrical pulse signal.

The counting circuit 67-n counts electrical pulse signals from the pulse height discrimination circuit 65-n at a readout cycle matching a view switching cycle. For example, a trigger signal TS is supplied from the control device 118 to the counting circuit 67-n at a switching timing of each view. In response to the supply of the trigger signal TS, the counting circuit 67-n adds 1 to a count number stored in an internal memory each time an electrical pulse signal is input from the pulse height discrimination circuit 65-n. In response to the supply of the next trigger signal, the counting circuit 67-n reads out data (that is, count data) of the count data accumulated in the internal memory and supplies the read data to the output circuit 69. The counting circuit 67-n resets the count number accumulated in the internal memory to an initial value each time the trigger signal TS is supplied. The counting circuit 67-n counts the count number for each view in this manner.

The output circuit 69 is connected to the counting circuits 67-n for a plurality of readout channels mounted on the X-ray detector 115. For each of a plurality of energy bins, the output circuit 69 integrates count data from the counting circuits 67-n for the plurality of readout channels to generate count data for the plurality of readout channels of each view. The count data of each energy bin is a set of data of count numbers specified by a channel, a segment (row), and an energy bin. The count data of each energy bin is transmitted to the console device 140 for each view. The count data for each view is called a count data set CS.

The DAS 116 having such a configuration collects count data indicating a count number (count value) of X-ray photons detected by the X-ray detector 115 for each set energy bin and acquires the count data of each energy bin as detection data.

The system control function 151 according to the second embodiment may set energy bins referenced by the reconstruction function 153 on the basis of an input operation received by the input interface 143, for example. The system control function 151 may automatically set energy bins without depending on an input operation received by the input interface 143.

The pre-processing function 152 according to the second embodiment generates projection data from the count data of each energy bin by performing prescribed pre-processing on the detection data (count data) output from the DAS 116. In other words, the pre-processing function 152 generates the projection data for each energy bin. The prescribed pre-processing may include, for example, logarithmic conversion processing, offset correction processing, inter-channel sensitivity correction processing, beam hardening correction, scattered ray correction, and dark count correction.

The reconstruction function 153 performs prescribed reconstruction processing on the projection data generated by the pre-processing function 152 to generate a CT image from the projection data.

For example, the reconstruction function 153 may generate a CT image (hereinafter, referred to as a bin image) of each energy bin by reconstructing the projection data for each energy bin.

The reconstruction function 153 may integrate the projection data of each energy bin, may calculate an amount of X-ray absorption on the basis of a total of the projection data and a response function stored in the memory 141, and may generate a counting image (also referred to as an integral image) on the basis of the amount of X-ray absorption.

Similarly to the first embodiment, the reconstruction function 153 may generate a CT image (basis material image) in which the basis material is enhanced, by extracting only a component of the basis material (for example, iodine) from the projection data of each energy bin and reconstructing the projection data of each energy bin from which the component of the basis material is extracted. The reconstruction function 153 may generate a virtual monochromatic X-ray image, a virtual simple image, an iodine map, an effective atomic number image, an electron density image, or the like, in addition to the basis material image.

The prescribed reconstruction processing may include filtered back projection method, a successive approximation reconstruction method, or the like. The reconstruction function 153 stores the reconstructed CT image in the memory 141. The reconstruction function 153 may perform the reconstruction processing using the detection data (count data) when the pre-processing is not performed by the pre-processing function 152.

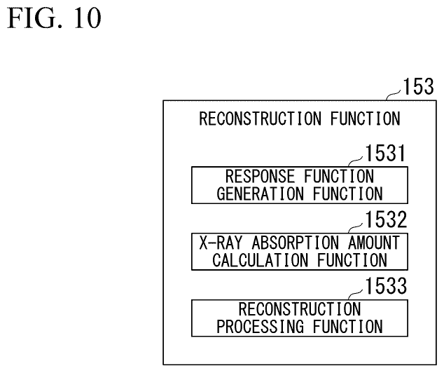

FIG. 10 is a diagram showing an example of a functional configuration of the reconstruction function 153 according to the second embodiment. The reconstruction function 153 has, for example, a response function generation function 1531, an X-ray absorption amount calculation function 1532, and a reconstruction processing function 1533.

The response function generation function 1531 generates data of a response function representing detector response characteristics. For example, the response function generation function 1531 measures a response (that is, detected energy and detected intensity) of a standard detection system to a plurality of monochromatic X-rays having a plurality of incident X-ray energies through predictive calculation, experiment, and a combination of predictive calculation and experiment, and generates a response function on the basis of measured values of the detected energy and the detected intensity. The response function generation function 1531 may generate data of a response function on the basis of actual measured values collected by calibration or the like. The response function specifies a relationship between detected energy of each incident X-ray and an output response of the system. For example, the response function specifies a relationship between detected energy and detected intensity of each incident X-ray. The generated data of the response function is stored in the memory 141.

The X-ray absorption amount calculation function 1532 calculates an X-ray absorption amount regarding each of a plurality of basis materials on the basis of the count data regarding a plurality of energy bins included in the projection data, an energy spectrum of the incident X-rays to the subject P, and the response function stored in the memory 141. The X-ray absorption amount calculation function 1532 can calculate the X-ray absorption amount with no influence of the response characteristics of the X-ray detector 115 and the DAS 116 by calculating the X-ray absorption amount on the basis of the count data and the energy spectrum of the incident X-rays to the subject P using the response function. In this way, the processing of obtaining the X-ray absorption amount for each basis material is also called material decomposition as described in the first embodiment. As the basis materials, all materials such as calcium, calcification, bone, fat, muscle, air, organs, lesions, hard tissues, soft tissues, and contrast materials can be set. The type of the basis material to be calculated may be determined in advance by the operator or the like via the input interface 143. The X-ray absorption amount indicates the amount of X-rays absorbed by the basis material. For example, the X-ray absorption amount is specified by a combination of an X-ray attenuation coefficient and an X-ray transmission path length.

The reconstruction processing function 1533 reconstructs a photo counting CT image that expresses a spatial distribution of the basis material to be imaged among a plurality of basis materials on the basis of the X-ray absorption amount regarding each of a plurality of basis materials calculated by the X-ray absorption amount calculation function 1532, and stores the generated CT image in the memory 141. The basis material to be imaged may be one type or may be a plurality of types. The type of the basis material to be imaged may be determined by the operator or the like via the input interface 143.

The projection data obtained by the photon counting CT apparatus includes information of the energy of the X-rays attenuated when the X-rays are transmitted through the subject P. For this reason, the reconstruction processing function 1533 can reconstruct, for example, a CT image of a specific energy component. Also, the reconstruction processing function 1533 can reconstruct, for example, a CT image of each of a plurality of energy components. In addition, the reconstruction processing function 1533 can assign, for example, a color tone according to an energy component to each pixel of a CT image of each energy component to superimpose a plurality of CT images color-coded according to the energy component.

The X-ray CT apparatus 100 that is a photo counting CT apparatus may generate a basis material image, a virtual monochromatic X-ray image, a virtual simple image, an iodine map, an effective atomic number image, or an electron density image through the reconstruction processing, in addition to or instead of generating the basis material image or the counting image through the reconstruction processing similarly to the first embodiment described above. In generating the basis material image or the counting image, the processing of FIGS. 3 and S102 of FIG. 4, that is, the material decomposition processing is omitted.

The training apparatus 200 according to the second embodiment prepares the same number of machine learning models MDL as the number of energy bin and trains each machine learning model MDL when the a bin image is generated for each energy bin by the X-ray CT apparatus 100. For example, when the number of energy bins is four, the number of machine learning models MDL is four. The training apparatus 200 prepares and trains one machine learning model MDL without depending on the number of energy bines when the counting image is generated by the X-ray CT apparatus 100.

According to the second embodiment described above, when the X-ray CT apparatus 100 is a photon counting CT apparatus, it is possible to train the machine learning model MDL using a training data set in which a basis material image, a virtual monochromatic X-ray image, a virtual simple image, an iodine map, an effective atomic number image, an electron density image, a bin image, or a counting image is employed as a target image.

### Other Embodiments

Hereinafter, other embodiments will be described. In the first or second embodiment described above, while a case where the processing circuitry 150 of the console device 140 of the X-ray CT apparatus 100 includes the pre-processing function 152, the reconstruction function 153, the image processing function 154, and the denoise processing function 155 has been described, the present disclosure is not limited thereto. For example, the training apparatus 200 or another external apparatus may include some or all of the pre-processing function 152, the reconstruction function 153, the image processing function 154, and the denoise processing function 155.

While a case where the processing circuitry 210 of the training apparatus 200 includes the generation function 214 and the machine learning function 216 has been described, the present disclosure is not limited thereto. For example, the processing circuitry 150 of the X-ray CT apparatus 100 may include one or both of the generation function 214 and the machine learning function 216.

In the X-ray CT apparatus 100 that is a photon counting CT apparatus, normal resolution (NR) data or super high resolution (SHR) data may be employed, in addition to or instead of a bin image, a counting image, a basis material image, a virtual monochromatic X-ray image, a virtual simple image, an iodine map, an effective atomic number image, or an electron density image.

In the above description, while a case where the same type of image is input to the machine learning model MDL in training and runtime (inference) has been described, the present disclosure is not limited thereto. For example, in training, the first machine learning model MDLa is trained on the basis of the first training data set in which the basis material image a added to the noise image of the basis material a is associated as a target with the synthetic image a. In this case, in runtime, the basis material image a is input to the first machine learning model MDLa. In this case, the first machine learning model MDLa may be used to obtain the denoise image from the basis material image b. On the contrary, the second machine learning model MDLb may be used to obtain the denoise image from the basis material image a. From the same viewpoint, a single machine learning model MDL that is trained using a CT image generated from projection data of monochromatic 70 keV may be applied to obtain denoise images from respective two basis material images of iodine and water.

A machine learning model MDL that is trained using projection data collected by different collection methods can also be used. For example, a single machine learning model MDL that is trained using an image of monochromatic 70 keV created from projection data collected by a DECT apparatus may be applied to three basis material images of iodine, water, and bone generated by a PCCT apparatus.

A machine learning model MDL that is trained using projection data collected by different detectors can also be used. For example, a machine learning model MDL that is trained using a counting image generated from projection data by an energy integral detector (EID) may be applied to a counting image created from projection data collected by a PCCT apparatus. In addition, the machine learning model MDL may also be applied to a spectral image such as a basis material image.

While certain embodiments have been described, the embodiments have been presented by way of example only, and are not intended to limit the scope of the invention. Indeed, the embodiments may be embodied in a variety of other forms; furthermore, various omissions, substitutions, and changes in the form of the embodiments may be made without departing from the spirit of the invention. The appended claims and equivalents thereof are intended to cover such forms or modifications as would fall within the scope and spirit of the invention.

What is claimed is:

1. A medical information processing apparatus comprising:
processing circuitry configured to
acquire projection data of X-rays with which a subject is irradiated,
generate a medical image from the projection data through reconstruction processing,
remove noise from the medical image using a trained model, and
output a denoise image that is the medical image with the noise removed, via an output interface,
wherein the trained model is a machine learning model that is trained on the basis of a training data set in which an output image including noise is associated as a target with an input image including noise.

2. The medical information processing apparatus according to claim 1,
wherein the processing circuitry is configured to
acquire a plurality of pieces of the projection data of different types,
generate, on the basis of a plurality of predetermined basis materials, basis material projection data that is the projection data corresponding to each of the plurality of basis materials, from the plurality of pieces of projection data through material decomposition processing, and
generate a prescribed type of medical image from the basis material projection data through the reconstruction processing.

3. The medical information processing apparatus according to claim 2,
wherein the prescribed type of medical image includes one of a basis material image, a virtual monochromatic X-ray image, a virtual simple image, an iodine map, an effective atomic number image, and an electron density image.

4. The medical information processing apparatus according to claim 2,
the medical image generated from the basis material projection data includes a first medical image and a second medical image,
the trained model includes a first trained model and a second trained model, and
noise is removed from the first medical image using the first trained model, and noise is removed from the second medical image using the second trained model.

5. The medical information processing apparatus according to claim 1,
wherein the processing circuitry is configured to
acquire the projection data generated for each energy bin from count data indicating a count number of photons of the X-rays, and
generate a prescribed type of medical image from the projection data generated for each energy bin.

6. The medical information processing apparatus according to claim 5,
wherein the prescribed type of medical image includes a bin image that is the medical image in which the projection data is reconstructed for each energy bin or a counting image that is the medical image in which the projection data of all energy bins are integrated and reconstructed.

7. The medical information processing apparatus according to claim 1,
wherein the input image included in the training data set is calculated on the basis of a difference between a first training image and a second training image,
the first training image is the medical image generated by reconstructing first subsampled data that is one subsampled data out of two pieces of subsampled data separated from training projection data as projection data to be trained by subsampling, and
the second training image is the medical image generated by reconstructing second subsampled data that is the other subsampled data out of the two pieces of subsampled data.

8. A medical information processing method using a medical image diagnostic apparatus, the medical information processing method comprising:
acquiring projection data of X-rays with which a subject is irradiated;
generating a medical image from the projection data through reconstruction processing;
removing noise from the medical image using a trained model; and
outputting a denoise image that is the medical image with the noise removed, via an output interface,
wherein the trained model is a machine learning model that is trained on the basis of a training data set in which an output image including noise is associated as a target with an input image including noise.

9. A computer-readable non-transitory storage medium storing a program, the program causing a computer to execute:
acquiring projection data of X-rays with which a subject is irradiated;
generating a medical image from the projection data through reconstruction processing;
removing noise from the medical image using a trained model; and
outputting a denoise image that is the medical image with the noise removed, via an output interface,
wherein the trained model is a machine learning model that is trained on the basis of a training data set in which an output image including noise is associated as a target with an input image including noise.
